(19) United States
(12) Patent Application Publication
(10) Pub. No.: US 2015/0293109 A1
(43) Pub. Date: Oct. 15, 2015

(54) Fluorescent Chemical Dye for Visualization of Neural Stem Cell Symmetric and Asymmetric Division

(71) Applicants: NATIONAL UNIVERSITY OF SINGAPORE, Singapore (SG); AGENCY FOR SCIENCE, TECHNOLOGY AND RESEARCH, Singapore (SG)

(72) Inventors: Young-Tae Chang, Singapore (SG); Seong-Wook Yun, Singapore (SG); Hyung Ho Ha, Singapore (SG)

(21) Appl. No.: 14/437,668

(22) PCT Filed: Oct. 28, 2013
(86) PCT No.: PCT/SG2013/000461

§ 371 Date: Apr. 22, 2015

Related U.S. Application Data
(60) Provisional application No. 61/719,587, filed on Oct. 29, 2012.

(51) Int. Cl. G01N 33/58; G01N 33/50; C07D 311/90

(52) U.S. Cl. CPC G01N 33/582; C07D 311/90; G01N 33/5058; G01N 2333/98

(57) ABSTRACT

A fluorescent rosamine dye having specificity for neural stem cells, specifically, proliferative neural stem cells, and represented by Structural Formula (I): wherein X is an anion, is described herein. Synthesis of the fluorescent rosamine dye and use of the fluorescent rosamine dye to detect symmetric and asymmetric division of neural stem cells are also described.

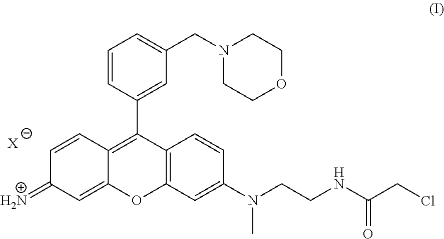

### RELATED APPLICATION

This application claims the benefit of U.S. Provisional Application No. 61/719,587, filed on Oct. 29, 2012. The entire teachings of the above application are incorporated herein by reference.

### BACKGROUND OF THE INVENTION

Although symmetric and asymmetric division of neural stem cells is a fundamental mechanism underlying brain development, the study of these cell divisions in real-time is still very difficult. Neural stem cells divide symmetrically for proliferation or asymmetrically for differentiation. Morphologically, these two types of division are indistinguishable.

The mouse neurosphere model system enables the study of mammalian brain development and neuronal disease research. Using nematode and fruit fly cells, which can be genetically manipulated to express fluorescent proteins fused to certain cell fate determinants, live imaging has been used to visualize different types of cell division. A few studies have shown the different types of divisions of neural stem cells in mouse and zebrafish brains by investigating the location and movement of fluorescent protein expressing cells. However, the imaging of the consecutive symmetric and asymmetric divisions of vertebrate cells in cell culture has not been possible due to the lack of proper markers and tools.

Thus, there is a need for a chemical dye that can be applied to cultured cells and visualized in real-time by, for example, fluorescence microscopy. Such a dye would be very useful in studying stem cell development and in identifying drugs that can modulate neural cell fate for use in regenerative medicine.

### SUMMARY OF THE INVENTION

A fluorescent rosamine dye that has specificity for neural stem cells is described herein. Synthesis of the fluorescent rosamine dye and application of the fluorescent rosamine dye to real-time optical imaging of symmetric and asymmetric division of neural stem cells are also described herein. The fluorescent rosamine dye is represented by Structural Formula (I):

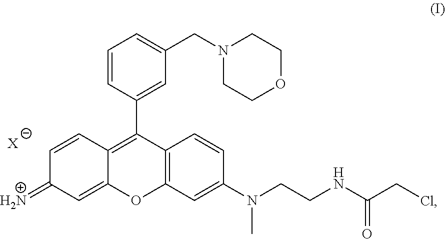

wherein X is an anion.

Also provided herein is a method of detecting a neural stem cell in a sample, the method comprising contacting a sample potentially containing a neural stem cell with a compound of Structural Formula (I) under conditions sufficient to enable the compound of Structural Formula (I) to label the neural stem cell, if present; and detecting a signal emitted by the compound of Structural Formula (I), thereby detecting the neural stem cell, if present, in the sample.

Also provided herein is a method of detecting symmetric and asymmetric division of a neural stem cell into a first daughter cell and a second daughter cell, the method comprising contacting a sample containing a neural stem cell with a compound of Structural Formula (I) under conditions sufficient to enable the compound of Structural Formula (I) to label the neural stem cell; allowing the neural stem cell to undergo division into the first daughter cell and the second daughter cell; and detecting a signal emitted by the compound of Structural Formula (I) in the first and second daughter cells, wherein signals of approximately equal intensities in the first and second daughter cells indicate symmetric division, and the presence of a substantially greater signal in the first daughter cell compared to the second daughter cell indicates asymmetric division, thereby detecting symmetric and asymmetric division of the neural stem cell into the first daughter cell and the second daughter cell.

Also provided herein is a method of identifying a compound that inhibits neural stem cell differentiation, the method comprising contacting a sample containing a neural stem cell with a compound of Structural Formula (I) and a compound that potentially inhibits neural stem cell differentiation under conditions sufficient to enable the compound of Structural Formula (I) to label the neural stem cell; incubating the neural stem cell under conditions sufficient to allow a neural stem cell that has not been contacted with the compound that potentially inhibits neural stem cell differentiation to undergo division into a first daughter cell and a second daughter cell; and detecting a signal emitted by the compound of Structural Formula (I), wherein a signal of substantially greater intensity in the sample treated with the compound that potentially inhibits neural stem cell differentiation compared to a control signal indicates inhibition of neural stem cell differentiation, thereby identifying a compound that inhibits neural stem cell differentiation.

Also provided herein is a method of identifying a compound that inhibits or stimulates neural stem cell differentiation, the method comprising contacting a first sample containing a neural stem cell with a compound of Structural Formula (I) and a compound that potentially inhibits or stimulates neural stem cell differentiation under conditions sufficient to enable the compound of Structural Formula (I) to label the neural stem cell; incubating the neural stem cell under conditions sufficient to allow a neural stem cell in a second sample that has not been contacted with the compound that potentially inhibits or stimulates neural stem cell differentiation to undergo division into at least a first daughter cell and a second daughter cell; and detecting a signal, if present, emitted by the compound of Structural Formula (I) in cells in the first and second samples, wherein a signal in a substantially different number of cells in the first sample than in the second sample indicates inhibition or stimulation of neural stem cell differentiation, thereby identifying a compound that inhibits or stimulates neural stem cell differentiation.

The compound of Structural Formula (I) stains a distinct neural stem cell population in mouse neurospheres, which are clusters of heterogeneous cells at various stages of differentiation. The specificity of the compound of Structural Formula (I) for this distinct neural stem cell population can be exploited to detect undifferentiated neural stem cells and to visualize both symmetric and asymmetric cell division by, for example, time lapse single cell imaging. Even distribution of the dye in the dividing cell indicates symmetric cell division, while uneven distribution of the dye in the dividing cell indicates asymmetric cell division.

The beta subunit of acid ceramidase was identified as the cellular binding target of CDy5 by a proteomics analysis. Neurosphere assay and single cell gene expression analysis showed that CDy5-stained cells are proliferative neural stem cells in which acid ceramidase expression is up-regulated. Furthermore, the formation of neurospheres is significantly inhibited by acid ceramidase inhibitors. This study highlights the importance of lipid metabolism in neurosphere cell proliferation and provides a valuable cell labeling tool for the study of the development of the mammalian central nervous system. The compound of Structural Formula (I) may also be a valuable tool for the study of the development of drugs for regenerative medicine.

### BRIEF DESCRIPTION OF THE DRAWINGS

The foregoing will be apparent from the following more particular description of example embodiments of the invention.

FIG. 1 is a confocal microscope image of a CDy5-stained mouse neurosphere (left panel, bright field image, scale bar=20 μm; right panel, fluorescence image taken using AIR (Nikon) confocal microscope).

FIG. 2 is a bar graph and shows the number of neurospheres generated from the same number of CDy5^{bright} and CDy5^{dim} cells as a function of cell passage number in a neurosphere assay for the assessment of neural stem cell selectivity of CDy5 (data represent average numbers of neurospheres in a culture dish with standard deviations).

FIGS. 3A and 3B show the symmetric (FIG. 3A) and asymmetric (FIG. 3B) division of a CDy5-stained neurosphere cell (CDy5-stained cells are indicated with white arrows) (scale bar=10 μm).

FIG. 4 are images from a long-term, time-lapse live imaging study of a CDy5-stained cell, and show neurosphere generation by both symmetric and asymmetric divisions (scale bar=10 μm). Cells remaining stained by CDy5 are marked with white arrows. Time format, hh:mm.

FIG. 5 are Z-stack three-dimensional (3D) confocal fluorescence micrographs, reconstructed for isosurfacing, of a neurosphere generated from a single CDy5-stained cell, and show that only two cells out of six remain stained by CDy5, as indicated with white arrows (left panel, whole morphology of a neurosphere with cytoplasm; right panel, only nuclei and CDy5-stained cytoplasm shown) (scale bar=5 μm).

FIG. 6 is a bar graph, and shows the results of a quantitative analysis of gene expression in 65 CDy5^{bright} and 69 CDy5^{dim} neurosphere cells by single-cell RT-PCR.

FIG. 7 is a bar graph and shows the number of neurospheres counted after six days of culture in medium containing the indicated concentration of CDy5 (data represent average numbers of neurospheres in a culture dish with standard deviations).

### DETAILED DESCRIPTION OF THE INVENTION

A description of example embodiments of the invention follows.

As used herein, “a,” “an” and “the” include plural referents unless the context clearly dictates otherwise. Thus, for example, reference to “a neural stem cell” can include a plurality of neural stem cells.

Provided herein is a fluorescent rosamine dye represented by Structural Formula (I):

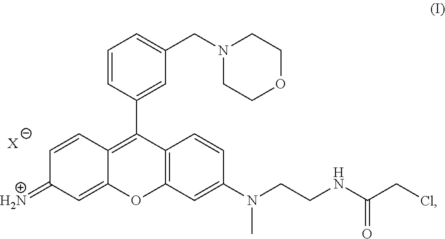

wherein X is an anion. Examples of anions are halide (e.g., fluoride, chloride, bromide, iodide), trifluoroacetate, acetate, benzenesulfonate, benzoate, perchlorate, sulfonate, bicarbonate, carbonate, citrate, mesylate, methylsulfate, nitrate, phosphate/diphosphate, and sulfate. The compound of Structural Formula (I) is also referred to herein as CDy5.

The compound of Structural Formula (I) is fluorescent. Therefore, the fluorescence signal emitted or produced by the compound of Structural Formula (I) can be detected using fluorescence microscopy. Fluorescence microscopy techniques are well-known in the art. For example, for single cell imaging, live cell imaging, live cell time-lapse imaging and clonal neurosphere imaging, a fluorescence microscope can be used to detect a signal emitted by a compound of Structural Formula (I), for example, a fluorescence signal emitted upon excitation of the compound of Structural Formula (I) using light of an appropriate wavelength. A fluorescence spectrometer, for example, a plate reader, can also be used to detect a signal emitted by a compound of Structural Formula (I), as can flow cytometry or fluorescence image analysis. Methods of the invention take advantage of the fact that the compound of Structural Formula (I) can be detected using microscopic techniques, such as fluorescence microscopy.

Provided herein is a method of detecting a neural stem cell in a sample, the method comprising contacting a sample potentially containing a neural stem cell with a compound of Structural Formula (I) under conditions sufficient to enable the compound of Structural Formula (I) to label the neural stem cell, if present; and detecting a signal emitted by the compound of Structural Formula (I), thereby detecting the neural stem cell, if present, in the sample. The signal is typically a fluorescence signal.

“Neural stem cell,” as used herein refers to a self-renewing, multipotent cell that generates the main phenotypes of the central nervous system. Typically, neural stem cells differentiate into neurons, astrocytes and oligodendrocytes.

A compound of Structural Formula (I) “labels” a neural stem cell if the compound of Structural Formula (I) binds to a component of the neural stem cell (e.g., a protein) with a dissociation constant (K_{d}) of less than about 10 μM. Preferably, the dissociation constant of binding is less than about 1 μM or, more preferably, less than about 100 nM. Binding can be measured by measuring a signal produced or emitted by the compound of Structural Formula (I), for example, upon excitation of the compound of Formula (I) with light. Alternatively, low angle static light scattering and particle size analysis can be used to detect binding of a compound of Formula (I) to a component(s) in a neural stem cell. Other methods suitable for measuring binding include nuclear magnetic resonance spectroscopy, X-ray crystallography and mass spectrometry.

A noticeable feature of CDy5 is that it has a chloroacetamide moiety which can form a covalent bond with a thiol group. Although not wishing to be bound by any particular theory, it is believed that CDy5 forms a covalent bond with a cysteine residue in the beta subunit of acid ceramidase (AC), a protein that hydrolyzes ceramide into fatty acid and sphingosine at a pH of about 4.5. However, as used herein, “binding” and “labeling” include both covalent and non-covalent interactions. In preferred embodiments, the compound of Structural Formula (I) covalently labels the neural stem cell, for example, by covalently binding to acid ceramidase. In other embodiments, the compound of Structural Formula (I) non-covalently labels the neural stem cell.

Thus, also provided herein is a method of detecting acid ceramidase in a sample, the method comprising contacting a sample potentially containing acid ceramidase with the compound of Structural Formula (I) under conditions sufficient to enable the compound of Structural Formula (I) to label acid ceramidase, if present; and detecting a signal emitted by the compound of Structural Formula (I), thereby detecting acid ceramidase, if present, in the sample.

Also provided herein is a method of detecting a cell expressing acid ceramidase in a sample, the method comprising contacting a sample potentially containing a cell expressing acid ceramidase with the compound of Structural Formula (I) under conditions sufficient to enable the compound of Structural Formula (I) to label the cell expressing acid ceramidase, if present; and detecting a signal emitted by the compound of Structural Formula (I), thereby detecting a cell expressing acid ceramidase, if present, in the sample. In some embodiments, the cell is a neural stem cell.

Typically, a signal, for example, a signal indicating the presence of a neural stem cell or acid ceramidase, is substantially greater than background signal. For example, a signal (e.g., a signal indicating the presence of a neural stem cell or acid ceramidase) can be at least two-fold greater than the intensity of background signal. Preferably, the intensity of the signal is at least five-fold, at least ten-fold, and, most preferably, at least fifty-fold greater than the intensity of background signal.

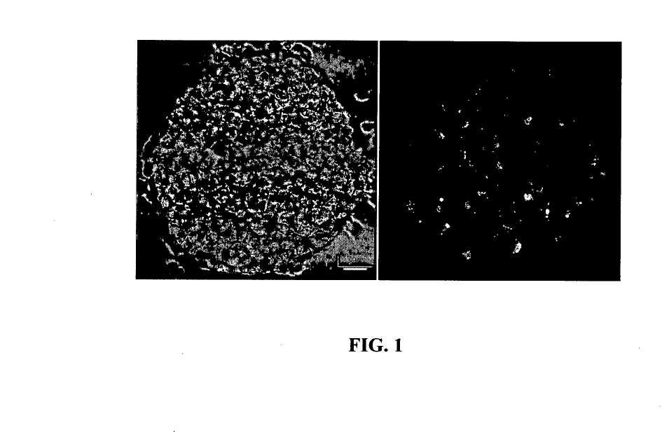

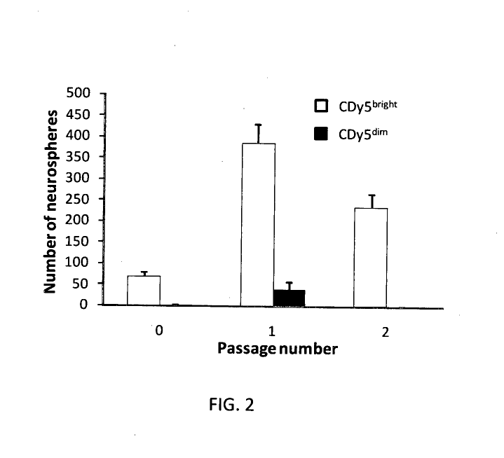

The method of detecting a stem cell in a sample can further comprise distinguishing between a neural stem cell and a differentiated neural cell in the sample. For example, when a mouse neurosphere was treated with CDy5, cytoplasmic staining of a distinct cell population within the neurosphere was observed (FIG. 1). The so-called CDy5^{bright} cells, those visible in the right panel of FIG. 1, emitted a signal that was substantially greater than the signal emitted by the so-called CDy5^{dim} cells. By collecting the CDy5^{bright} cells and the CDy5^{dim} cells by fluorescence-activated cell sorting (FACS), and subsequently culturing the CDy5^{bright} cells separately from the CDy5^{dim} cells, it was determined that CDy5^{bright} cells make more than 10 times more neurospheres than CDy5^{dim} cells, suggesting that CDy5 stains neural stem or progenitor cells more strongly than differentiated neural cells (FIG. 2). In the experiment described above, a cell was considered to be a CDy5^{dim} cell if no signal could be detected by fluorescence microscopy, or the signal detected was not substantially greater than background signal.

“Differentiated neural cell,” as used herein, refers to a cell that is a progeny, for example, a daughter cell, of a neural stem cell. Differentiated neural cells can be produced by the asymmetric division of a neural stem cell into two daughter cells. Differentiated neural cells include neurons, astrocytes and oligodendrocytes.

The method of distinguishing between a neural stem cell and a differentiated neural cell comprises contacting a sample containing a neural stem cell and a differentiated neural cell with a compound of Structural Formula (I) under conditions sufficient to enable the compound of Structural Formula (I) to label the neural stem cell; and detecting a signal emitted by the compound of Structural Formula (I), wherein the presence of a signal indicates a neural stem cell, thereby distinguishing between the neural stem cell and the differentiated neural cell.

The fact that CDy5 stains neural stem or progenitor cells more strongly than differentiated neural cells can also be used to visualize symmetric and asymmetric cell divisions. Symmetric and asymmetric cell divisions are the most fundamental mechanisms of the development of a multi-cellular organism from a zygote (1,5). A neurosphere is a particularly interesting material to study the two different types of cell division because a neural stem cell can grow within a week to a neurosphere composed of thousands of cells at various stages of differentiation. It is known that a small number of cells in a neurosphere remain as stem cells by symmetric divisions, but the majority of the cells are differentiated cells generated by asymmetric divisions (39). Although neurospheres provide a good model system to investigate brain development and neural stem cell therapy, research has been hampered by the lack of proper cellular markers and tools to distinguish stem cells and differentiated cells in living neurospheres (40).

Therefore, provided herein is a method of detecting symmetric and asymmetric division of a neural stem cell into a first daughter cell and a second daughter cell, the method comprising contacting a sample containing a neural stem cell with a compound of Structural Formula (I) under conditions sufficient to enable the compound of Structural Formula (I) to label the neural stem cell; allowing the neural stem cell to undergo division into the first daughter cell and the second daughter cell; and detecting a signal emitted by the compound of Structural Formula (I) in the first and second daughter cells, wherein signals of approximately equal intensities in the first and second daughter cells indicate symmetric division, and the presence of a substantially greater signal in the first daughter cell compared to the second daughter cell indicates asymmetric division, thereby detecting symmetric and asymmetric division of the neural stem cell into the first daughter cell and the second daughter cell.

In some embodiments, the division is symmetric division. In other embodiments, the division is asymmetric division.

The methods described above can also comprise detecting the signal emitted by the compound of Structural Formula (I) using live-cell imaging, for example, single-cell live-cell imaging.

Also provided herein is a method of identifying a compound that inhibits neural stem cell differentiation, the method comprising contacting a sample containing a neural stem cell with a compound of Structural Formula (I) and a compound that potentially inhibits neural stem cell differentiation under conditions sufficient to enable the compound of Structural Formula (I) to label the neural stem cell; incubating the neural stem cell under conditions sufficient to allow a neural stem cell that has not been contacted with the compound that potentially inhibits neural stem cell differentiation to undergo division into a first daughter cell and a second daughter cell; and detecting a signal emitted by the compound of Structural Formula (I), wherein a signal of substantially greater intensity in the sample treated with the compound that potentially inhibits neural stem cell differentiation compared to a control signal indicates inhibition of neural stem cell differentiation, thereby identifying a compound that inhibits neural stem cell differentiation.

As used herein, “control signal” refers to a signal that is representative of a sample (e.g., comprising a neural stem cell) that has not been subjected to the experimental condition being tested. The sample used to obtain the control signal should be otherwise substantially equivalent to the sample being subjected to the experimental condition being tested. For example, a control signal can be obtained by contacting a sample containing a neural stem cell with a compound of Structural Formula (I) and a vehicle, such as DMSO, under conditions sufficient to label the neural stem cell; incubating the neural stem cell under conditions sufficient to allow the neural stem cell to undergo division into a first daughter cell and a second daughter cell; and detecting the signal emitted by the compound of Structural Formula (I).

Also provided herein is a method of identifying a compound that inhibits or stimulates neural stem cell differentiation, the method comprising contacting a first sample containing a neural stem cell with a compound of Structural Formula (I) and a compound that potentially inhibits or stimulates neural stem cell differentiation under conditions sufficient to enable the compound of Structural Formula (I) to label the neural stem cell; incubating the neural stem cell under conditions sufficient to allow a neural stem cell in a second sample that has not been contacted with the compound that potentially inhibits or stimulates neural stem cell differentiation to undergo division into at least a first daughter cell and a second daughter cell; and detecting a signal, if present, emitted by the compound of Structural Formula (I) in cells in the first and second samples, wherein a signal in a substantially different number of cells in the first sample than in the second sample indicates inhibition or stimulation of neural stem cell differentiation, thereby identifying a compound that inhibits or stimulates neural stem cell differentiation. In some embodiments, the method is a method of identifying a compound that inhibits neural stem cell differentiation. In other embodiments, the method is a method of identifying a compound that stimulates neural stem cell differentiation.

In some embodiments of the methods of identifying a compound that inhibits or inhibits or stimulates neural stem cell differentiation, the method comprises detecting the signal emitted by the compound of Structural Formula (I) using flow cytometry or fluorescence image analysis. Using flow cytometry or fluorescence image analysis, the signal of the entire sample can be detected or the number of cells emitting a signal can be counted.

The methods of detecting symmetric and asymmetric division of a neural stem cell and identifying a compound that inhibits neural stem cell differentiation can further comprise stimulating neural stem cell differentiation by contacting the neural stem cell with an agent that stimulates neural stem cell differentiation. Examples of agents that stimulate neural stem cell differentiation are known in the art, and include brain-derived neurotrophic factor and retinoic acid.

The methods described herein can further comprise substantially removing or removing unbound (e.g., excess) compound of Structural Formula (I) from the sample. Preferably, unbound compound of Structural Formula (I) is substantially removed or removed from the sample prior to detecting a signal emitted by the compound of Structural Formula (I), if present.

“Substantially removing,” as used herein, refers to removing enough of the unbound compound of Structural Formula (I) such that its presence does not interfere with or materially alter the detection of the signal of bound compound of Structural Formula (I). For example, in methods involving detection of a cell expressing AC, unbound compound of Structural Formula (I) may be considered to be substantially removed if its presence does not result in a false positive detection of a cell expressing AC.

Unbound compound of Structural Formula (I) can be removed from a sample, for example, by removing culture medium containing the compound of Structural Formula (I) from a cell in a sample, with or without rinsing the cell. Other methods of removing unbound compound of Structural Formula (I) from a sample are known to those of ordinary skill in the art.

### EXEMPLIFICATION

Development of a multicellular organism from a single-cell zygote requires precisely orchestrated symmetric and asymmetric cell divisions. Two daughter cells with different fates are produced by an asymmetric division to generate cellular diversity, while identical daughter cells are produced by a symmetric division to proliferate. In the case of stem cells, which have the capability to self-renew and give rise to multiple types of cells, at least one daughter cell must retain the properties of the mother cell (1). During asymmetric division, cells are polarized and certain cellular components are segregated into one half of the cell resulting in an uneven distribution of the components between two daughter cells (2). A neurosphere generated from a mouse neural stem cell is a particularly interesting material to study the two different types of cell division in mammals. A single neural stem cell can grow within a week to a neurosphere composed of hundreds of cells at various stages of differentiation (3). It is known that small numbers of cells in a neurosphere remain as stem cells by symmetric divisions but a majority of the cells are differentiated cells produced by asymmetric divisions (4).

Synthesis of CDy5

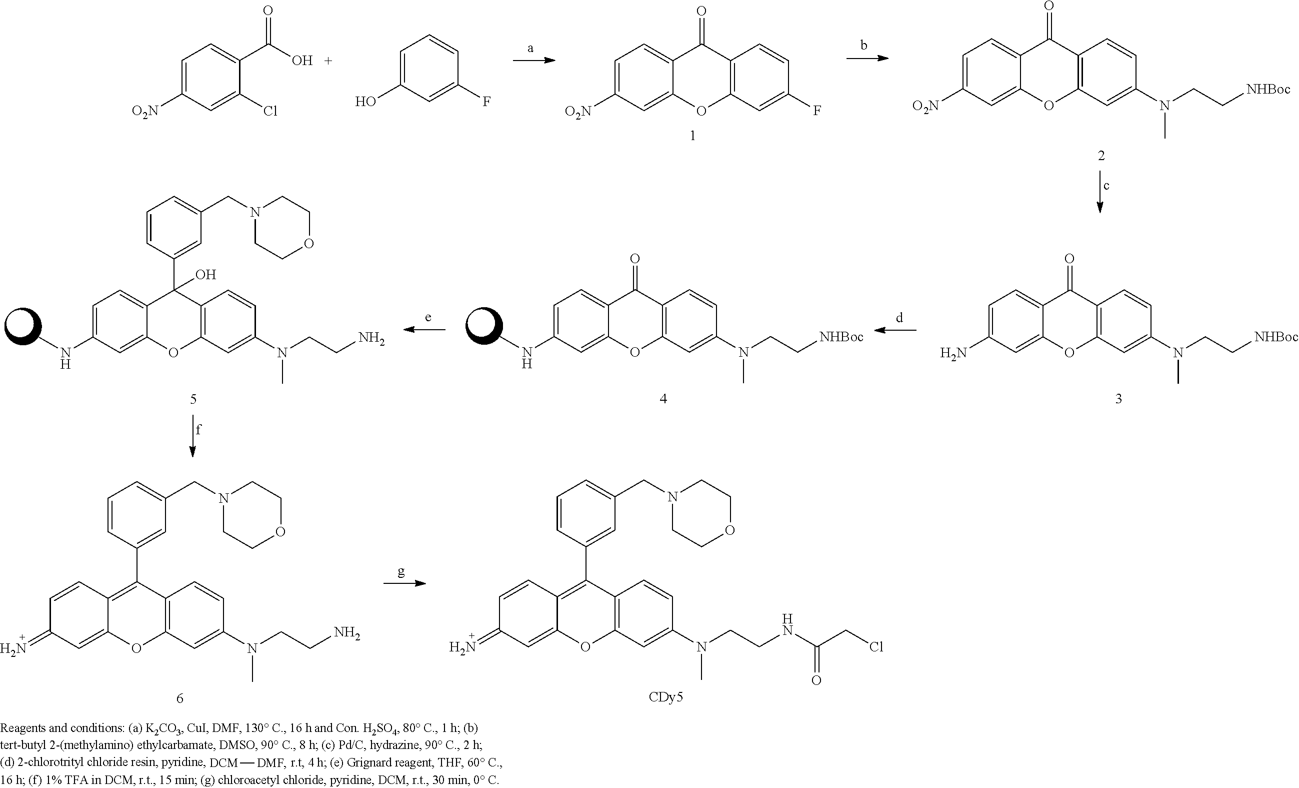

Compound 1—

To a solution of 2-chloro-4-nitrobenzoic acid (3.0 g, 14.88 mmol) in DMF (40 mL) was added 3-fluorophenol (2.47 g, 16.38 mmol), potassium carbonate (3.08 g, 16.38 mmol) and copper powder (102 mg, 1.61 mmol). After heating at 130° C. overnight, the reaction mixture was cooled to room temperature, and slowly poured into ice-cold 1N HCl solution (300 mL). The solution was stirred until a brown solid was formed. The solid was filtered and washed with cold water to yield a brown solid (3.1 g). The crude solid was dissolved in conc. sulfuric acid (20 mL), and heated at 80° C. for 1 hr. After cooling to room temperature, the reaction mixture was poured into ice (350 mL) and stirred for 1 h. The precipitated solid was filtered, washed with cold water and dried under vacuum to render Compound 1. ¹H-NMR (CDCl3) δ 8.50 (d, J=8.48, 1H), 8.37 (m, 2H), 8.20 (dd, J=1.75, 1H), 7.21 (m, 2H) MS (ESI): m/z 260.18 (M+1).

Compound 2—

Compound 1 (1.0 equivalent) was dissolved in DMSO (0.2 M) and the tert-butyl 2-(methylamino)ethylcarbamate amine was added (2 equivalents) in one portion. The reaction mixture was heated to 90° C. and stirred for 8 hours. The solution was then cooled to room temperature and water was added. The precipitate was collected and washed with diethyl ether and water to render Compound 2. ¹H-NMR (CDCl3) δ 8.42 (d, J=8.48, 1H), 8.23 (d, J=1.75, 1H), 8.11 (dd, J=9.06, 2.04, 2H), 6.80 (dd, J=2.04, 1H), 6.55 (d, J=2.05, 1H), 4.79 (br, NH), 3.62 (m, 2H), 3.36 (m, 2H), 3.12 (s, 3H), 1.42 (s, 9H) MS (ESI): m/z 414.42 (M+1).

Compound 3—

A solution of Compound 2 (826 mg, 2 mmol) in ethanol (20 mL) was purged with nitrogen for 10 min. Hydrazine monohydrate (0.485 mL, 10 mmol) and 10% Pd/C (83 mg) were added. The mixture was refluxed under nitrogen for 2 hrs. Then, after removing Pd/C by filtration, the crude mixture was concentrated under vacuum to yield Compound 3, which was used without further purification in the reaction with 2-chloro-trityl chloride resin.

Compound 4—

Compound 3 (0.75 mmol) was dissolved in dichloromethane (10 mL) and added to 2-chlorotrityl chloride resin (0.5 mmol) suspended in dichloromethane (1 mL) and pyridine (3 mmol). After stirring for 4 hours, the resin was filtered and washed with DMF (×5), methanol (×10), and dichloromethane (×10), and dried under vacuum to provide Compound 4.

Compound 6—

To a solution of Compound 4 (100 mg, 0.1 mmol) in freshly distilled THF (5 mL), a solution of [3-(4-morpholinylmethyl)phenyl] magnesium bromide 0.25M was added and shaken overnight at 60° C. The resin was filtered and washed with dichloromethane (×5), DMF (×5), methanol (×5), and dichloromethane (×5). The resin was then dried and treated with 1% TFA in dichloromethane (3 mL) for 15 minutes. The filtrate containing Compound 6 was collected and used in the next step without further purification.

CDy5—

Compound 6 was dissolved in dichloromethane (4 mL) and cooled in an ice bath. To the solution was added pyridine (0.5 mL), followed by chloroacetic anhydride (100 mg). After 30 minutes, the reaction mixture was diluted with DCM, washed sequentially with 1N HCl, aq. NaHCO₃, and brine, dried over anhydrous sodium sulfate, concentrated, and purified by silica gel column chromatography to give CDy5 (5 mg, 0.047 mmol). ¹H-NMR (MeOH-d₄) δ 9.37 (br s, NH), 7.82 (m, 2H), 7.65 (s, 1H), 7.59 (d, J=7.02 Hz, 1H), 7.31 (m, 2H), 7.13 (m, 2H), 6.89 (s, 1H), 6.86 (s, 1H), 5.33 (NH, 1H), 4.49 (s, 2H), 3.96 (m, 3H), 3.81 (m, 3H), 3.55 (t, J=6.14 Hz, 2H), 3.34 (m, 5H)¹³C-NMR (MeOH-d₄) δ 161.62, 160.06, 159.02, 158.61, 158.24, 134.52, 134.12, 133.38, 133.23, 132.52, 132.26, 130.80, 118.27, 115.47, 114.90, 114.21, 98.45, 97.85, 64.78, 61.16, 52.82, 52.26, 44.71, 42.87, 39.60, 38.12 ppm. HRMS (ESI): Calculated for C₂₉H₃₁ClN₄O₃(M+H)⁺: 519.03 Found: 519.2179.

### Neurosphere Preparation

Mouse brains harvested from E14.5 fetuses were digested with 0.25% trypsin/1mM EDTA solution for 30 minutes at 37° C. The tissues were triturated sequentially with a 10-mL pipette, a 1-mL pipette and a 0.2-mL pipette in medium containing 10% fetal bovine serum (FBS). The dissociated cells were washed 3 times with PBS by repeated resuspension and centrifugation, and filtered through a 40-μm strainer. The obtained single cells were plated in DMEM/F12 medium containing 10 ng/mL bFGF, 20 ng/mL EGF and B27 without vitamin A, and cultured for 7-10 days without changing the medium.

### CDy5 Stains Stem Cells in Neurospheres

Dissociated neurosphere cells were cultured in a complete neurosphere culture medium containing 2 μM CDy5 for six days. They were dissociated for single cell imaging, live cell time-lapse imaging and clonal neurosphere imaging. For imaging at a later time, the neurospheres were fixed with 4% paraformaldehyde for 5 minutes and stored in PBS at 4° C.

FIG. 1 is a confocal microscope image of a CDy5-stained mouse neurosphere (left panel, bright field image, scale bar=20 μm; right panel, fluorescence image taken using A1R (Nikon) confocal microscope) and shows that a distinct cell population in the neurospheres was selectively stained by CDy5.

To examine the relationship between stemness and CDy5 staining, CDy5^{bright} and CDy5^{dim} neurosphere cells were sorted and collected by FACS and separately resuspended in neurosphere culture medium and plated in triplicate in E-well culture plates at a density of 3,000 cells per well. The cells were then cultured in an incubator without agitation. After six days of culture, the number of neurospheres was counted manually under a microscope.

FIG. 2 is a bar graph of the number of neurospheres generated from the same number of CDy5^{bright} and CDy5^{dim} cells, and shows that CDy5^{bright} cells generated more than ten times more neurospheres than CDy5^{dim} cells in three independent experiments conducted with cells of different passage numbers, suggesting that CDy5 stains neural stem or progenitor cells more strongly than differentiated cells.

To determine the percentages of neurospheres generated from CDy5^{bright} and CDy5^{dim} cells possessing different differentiation potentials, neurospheres generated from CDy5^{bright}(n=48 and 34) and CDy5^{dim} (n=43 and 43) cells were classified into tri-, bi- and uni-potent cells, depending on the number of positively stained cell types. Single neurospheres were plated onto glass coverslips coated with laminin and poly-L-lysine and cultured in bFGF/EGF-depleted neurosphere culture medium containing 5% fetal bovine serum. The differentiated cells were fixed with 4% paraformaldehyde and stained using primary antibodies: Tuj1 (Covance), glial fibrillary acidic protein (GFAP) (Dako), and O4 (Millipore), and appropriate secondary antibodies: Alexa Fluor 488 goat anti-mouse, Alexa Fluor 594 goat anti-mouse and Alexa Fluor 647 donkey anti-rabbit (Life Technologies), respectively. Tuj1 was used as a marker for neurons, GFAP was used as a marker for astrocytes and 04 was used as a marker for oligodendrocytes.

When the neurospheres were randomly differentiated in a medium containing only FBS, and immunostained against astrocyte, neuron and oligodendrocyte markers as described above, more numbers of neurospheres generated from CDy5^{bright} cells differentiated into all three types of cells than those from CDy5^{dim} cells. The CDy5^{bright} cells differentiated into 30±4% uni-potent cells, 26±1 bi-potent cells and 44±3 tri-potent cells. The CDy5^{dim} cells differentiated into 47±6 uni-potent cells, 22±1 bi-potent cells and 31±7 tri-potent cells. Data represent mean±standard deviation (SD) of duplicates. These data suggest that CDy5 selectively stains proliferative neural stem cells in heterogeneous cell populations of different stages of differentiation.

To determine if CDy5 forms a covalent bond with a protein, neurospheres stained with CDy5 and Hoechst 33342 were fixed using 4% paraformaldehyde followed by absolute methanol, which extracts organic dyes bound to their targets by noncovalent bonding. Both CDy5 and Hoechst signals were detected when observed after fixation with paraformaldehyde. However, following methanol treatment, Hoechst 33342 was almost completely washed out, while CDy5 remained without losing its signal intensity. Because methanol makes cell membranes permeable and extracts weakly bound small molecules from cells, this result suggests that CDy5 binds to a protein and forms a covalent bond. Although not wishing to be bound by any particular theory, this indicates that CDy5 binds to a protein that is more highly expressed in stem cells than in differentiated cells and forms a covalent bond with a cysteine nearby the binding site.

### CDy5 can be Used to Visualize Symmetric and Asymmetric Cell Division

The stem cell specificity of CDy5 and its strong binding to a protein led to the hypothesis that CDy5 might be useful for imaging symmetric and asymmetric distribution of its target protein during cell division. For time-lapse imaging of single cells, CDy5-stained neurospheres were dissociated into single cells. Brightly stained cell images were periodically acquired using a microscope equipped with a cell incubator system. During this imaging, CDy5 was not added again.

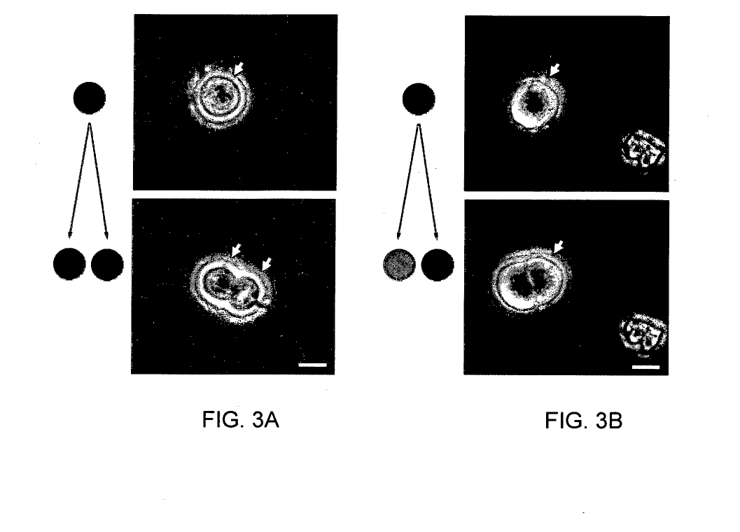

The acquired phase contrast images showed single cell divisions which gave rise to two morphologically identical daughter cells. Fluorescence images acquired in parallel showed even distribution of CDy5 in some cell divisions and uneven distribution in other cell divisions, reflecting symmetric and asymmetric divisions. FIGS. 3A and 3B show the symmetric (FIG. 3A) and asymmetric (FIG. 3B) division of a CDy5-stained neurosphere cell (CDy5-stained cells are indicated with white arrows).

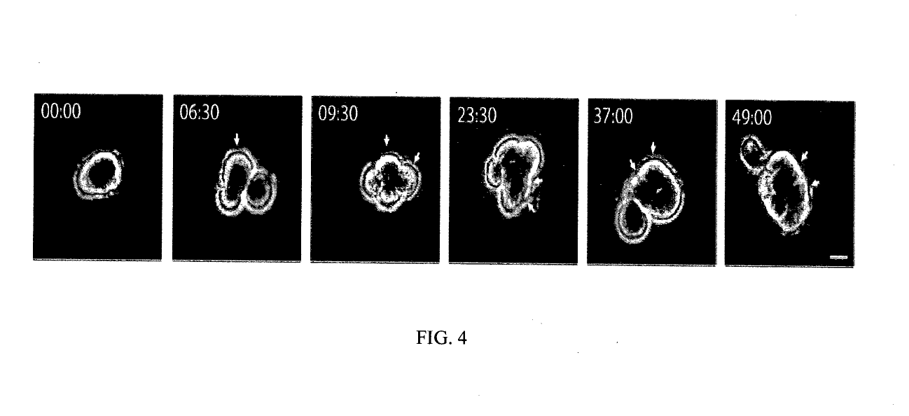

Long-term image acquisitions for two consecutive days showed the growth of CDy5-stained single cells into multi-cell neurospheres by both symmetric and asymmetric divisions (FIG. 4). Prolonged image acquisition for two days showed restricted distribution of CDy5 during the growth of CDy5-stained cells into multi-cell neurospheres by further cell divisions. Thus, CDy5-stained cells can be identified in a neurosphere even after multiple cell divisions.

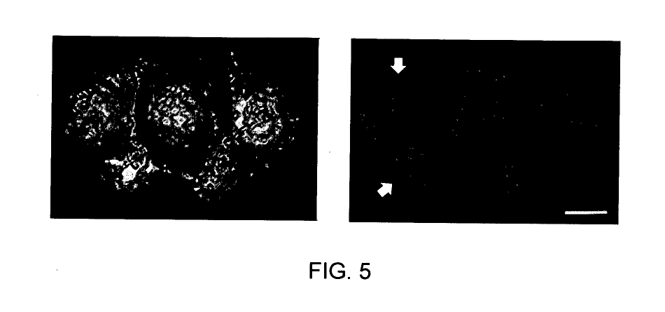

Confocal 3D imaging of a neurosphere generated from a single CDy5-stained cell also reinforced the phenomenon observed in FIG. 4. FIG. 5 are 3D images reconstructed from z-stack confocal images of a multi-cell neurosphere generated from a single CDy5-stained cell. FIG. 5 shows that only two cells out of six remain stained by CDy5, indicated with white arrows (left panel, whole morphology of a neurosphere with cytoplasm; right panel, only nuclei and CDy5-stained cytoplasm shown). The cells that remained stained by CDy5 had the morphological structure of a whole neurosphere.

### CDy5 Binds to Acid Ceramidase (AC)

Neurosphere proteins were analyzed to identify the cellular binding target of CDy5 by a proteomics approach. Neurospheres stained with CDy5 were collected by centrifugation at 453×g for 3 minutes and the pellet was washed three times with cold PBS before resuspension in a lysis buffer containing 40 mM Tris, 7 M urea, 2 M thiourea, 4% CHAPS (Sigma), 104/mL protease inhibitor cocktail (EDTA free, GE healthcare), 50 μg/mL DNase I and 50 μg/mL RNase A. For extraction of only cytosolic soluble proteins, the cells were lysed in a buffer containing 40 mM Tris, protease inhibitors cocktail, DNase I and RNase A. The cell extract was homogenized by ultrasonication for 30 seconds and then incubated for 30 minutes at room temperature. The supernatant was collected after centrifugation at 20,000×g for 45 minutes at 4° C. The protein concentration was determined by Bradford protein assay reagent (Bio-Rad).

The protein sample of 1 mg was diluted in 340 μL at of rehydration buffer containing 7 M urea, 2 M thiourea, 4% CHAPS, 20 mM DTT and 0.5% IPG buffer (GE healthcare), and loaded to 18 cm ReadyStrip™ IPG strips pH 3-10NL or pH 5-8NL (Bio-Rad) by passive rehydration. It was separated first by isoelectric focusing for 60,000 Vhrs at 20° C. on a PROTEAN IEF Cell (Bio-Rad). Then, the IEF strips were reduced in an equilibration buffer I containing 50 mM Tris-HCl (pH 8.8), 6 M urea, 30% glycerol, 2% SDS and 2% DTT at room temperature for 10 minutes and alkylated with a SDS-PAGE equilibration buffer II containing 50 mM Tris-HCl (pH 8.8), 6 M urea, 30% glycerol, 2% SDS, 2.5% iodoacetamide and a trace of bromophenol blue at room temperature for an additional 10 minutes. The equilibrated IEF strips were embedded in 0.5% low melting temperature agarose dissolved in Tris-glycine-SDS buffer on top of the second dimension 12% SDS-PAGE gel. After electrophoresis for 5 hours at 30 mA, the gel was scanned on a Typhoon 9400 scanner (GE healthcares) for two-dimensional fluorescence image. A duplicate gel was stained using PlusOne™ Silver Staining Kit (GE healthcare) according to the manufacturer's protocol.

When the proteins extracted from CDy5-stained neurospheres were separated by two-dimensional SDS-PAGE and scanned on a fluorescence scanner as described above, three major fluorescent spots of about 35 kDa were detected among many different proteins detected by silver staining. These spots were excised from the gel for in-gel tryptic digestion and peptide extraction. The excised gel was washed with water and cut into small pieces of approximately 1 mm³ in size. The gel pieces were rinsed with 50% acetonitrile/25 mM ammonium bicarbonate buffer (pH 7.8) three times, dehydrated in 100% acetonitrile and dried by speed vac. They were digested with 10 ng/pt trypsin gold, (Mass Spectrometry grade, Promega) in 25 mM ammonium bicarbonate buffer (pH 8.0) overlaid with 10 μL of 25 mM ammonium bicarbonate buffer for 16 hours at 37° C. The peptides were extracted sequentially with 20 mM ammonium bicarbonate buffer followed by 50% acetonitrile in 0.1% trifluoroacetic acid (TFA). The pooled peptides were dried using a speed vac and dissolved in 7 μL of 0.1% TFA.

LC MALDI-TOF/TOF mass spectrometry analysis of the peptide sample provided a list of candidate proteins, including protein phosphatase 1 gamma catalytic subunit, (PP1γ) and N-acylsphingosine amidohydrolase (acid ceramidase; AC) β subunit whose molecular weights are about 35 kDa. Specifically, tryptic peptides in 6.4 μL were injected into Dionex Ultimate 3000 capillary HPLC system equipped with Acclaim® PepMap™ μ-Guard columns. Column temperature was maintained at 25° C., micropump flow rate was 4 μL/min and acetonitrile gradient from 5 to 60% in 0.05% TFA was applied within 1 hour. Fractions (10 sec/spot) were directly spotted onto Prespotted AnchorChip target plate 384 with LC coupled Proteineer Fc (Bruker Daltonics) according to manufacturer's protocol. The peptide MALDI MS and MS/MS was analysed using UltraFlex III TOF-TOF (Bruker Daltonics) with WarpLC 1.2 and the Compass 1.2 software package including FlexControl 3.0 and FlexAnalysis 3.0 with PAC peptide calibration standards. The peak lists were submitted to an in-house Mascot server using BioTools 3.2 (Bruker Daltonics) and searched against SwissProt database (517100 sequences) with peptide mass tolerance of 100 ppm and one missed cleavage allowed, considering for variable modifications of carbamidomethyl of cysteine and oxidation of methionine.

By a two-color fluorescence two-dimensional Western blot analysis, AC was determined to be the protein that binds to CDy5. A protein sample of 1.5 mg extracted from CDy5-stained neurosphere was separated by 2D SDS-PAGE. The proteins were transferred from a part of the gel (5×8 cm) containing the major fluorescence spots onto a PVDF membrane. The membrane was blocked with PBS containing 0.05% Tween 20 and 5% skim milk for 1 hour and incubated with goat anti-acid ceramidase polyclonal antibody (T-20) (1:500 dilution, Santa Cruz, sc-28486), which was detected using donkey anti-goat IgG-Alexa 647. Fluorescent signals from CDy5 and the antibody were detected on a Typhoon 9.4 scanner and analyzed using ImageQuant 5.2 software (GE healthcare).

The results of the two-dimensional Western blot analysis were confirmed by pull-down assay. The cytosolic soluble protein sample of 1 mg extracted from CDy5-stained neurosphere was adjusted to pH 7.5 and a concentration of 2 mg/mL with 1N HCl for a final volume of 0.5mL. It was mixed 1:1 in volume:volume with 2×IP buffer containing 2% Triton X-100, 300 mM NaCl, 2 mM EDTA, 1% NP-40, 0.2% SDS, 10 mM DTT and 2× protease inhibition cocktail, and then heated at 95° C. for 2 minutes. The supernatant obtained by centrifugation was incubated with 2 μg goat anti-acid ceramidase antibody at 4° C. overnight with agitation. The sample was incubated with 1.5 mg of Protein G Dynabeads (Invitrogen) at 4° C. for 2 hours on a rotating mixer and then washed with IP buffer followed by PBS and 0.15 M NaCl containing protease inhibitor. The protein was eluted in 30 μL of 2× Laemmli buffer by heating at 95° C. for 5 minutes and subjected to 12% SDS-PAGE. Fluorescent signals from CDy5 were detected on a Typhoon 9.4 scanner. The pull-down assay showed strengthened CDy5 signal intensity in a sample pulled down by AC antibody but not by PP1γ antibody.

AC is synthesized as a precursor polypeptide of 395 amino acids in human (13) and 394 amino acids in mouse (14), which is processed into nonglycosylated α subunit and glycosylated β subunit (15). As the mouse 13 subunit of 253 amino acids has five potential N-glycosylation sites, CDy5-stained neurosphere cell lysate was treated with peptide-N-glycosidase (PNGase) F, which removes N-glycan from the protein. This resulted in a downward shift of the fluorescent band from about 35 kDa to about 25 kDa due to a faster migration of the deglycosylated protein in SDS-PAGE, further confirming that the fluorescence signal is from the CDy5 bound to the β subunit of AC. Furthermore, MS/MS fragment analysis revealed that CDy5 binds to the first N-terminal amino acid residue cysteine of AC β subunit.

Having found that CDy5 preferably stains proliferative neural stem cells in neurosphere by binding to AC, the expression levels of Asah1 and 38 other genes associated with neural stem cell and its differentiation (4,16) in CDy5^{bight} and CDy5^{dim} neurosphere cells was examined by single cell quantitative RT-PCR. Individual CDy5^{bright and dim} cells were sorted by FACS and collected directly into 96-well plate containing 10 μl of RT-PreAmp master mix containing 5 μL CellsDirect 2× reaction mix (Invitrogen), 2.50, 0.2× assay pool (Applied Biosystems), 0.5 μL, SuperScript® III RT/Platinum® Taq mix (Invitrogen) and 2 μL TE buffer (Qiagen) per well. Cells were frozen at −80° C. and thawed to induce lysis. cDNAs were generated from sequence-specific reverse primers by a reverse transcription at 50° C. for 20 minutes followed by enzyme inactivation at 95° C. for 2 minutes. The cDNA was pre-amplified by 18 cycles of denaturation at 95° C. for 15 seconds and annealing/synthesis at 60° C. for 4 minutes. These pre-amplified RT-PCR products were quantified by real-time PCR using a 48.48 dynamic array (Fluidigm) on the BioMark™ System (Fluidigm). Ct values higher than 28 were considered undetectable and the value of 28 was used as its Ct for calculation in such cases. In total, data for 48 genes including house-keeping gene β-Actin in 96 CDy^{bright} and 96 CDy5^{dim} cell samples were obtained. Data from the cells in which β-Actin expression was not detectable or above mean±3×SD and the genes whose expressions were not detectable in more than 50% of the cells both in CDy5^{bright and dim} groups were excluded from the statistical analysis. Finally, 39 gene expressions in 65 CDy^{bright} and 69 CDy5^{dim} cells were analyzed. Ct values of the gene of interest for a specific cell were normalised by subtracting the Ct value of β-Actin for the same cell.

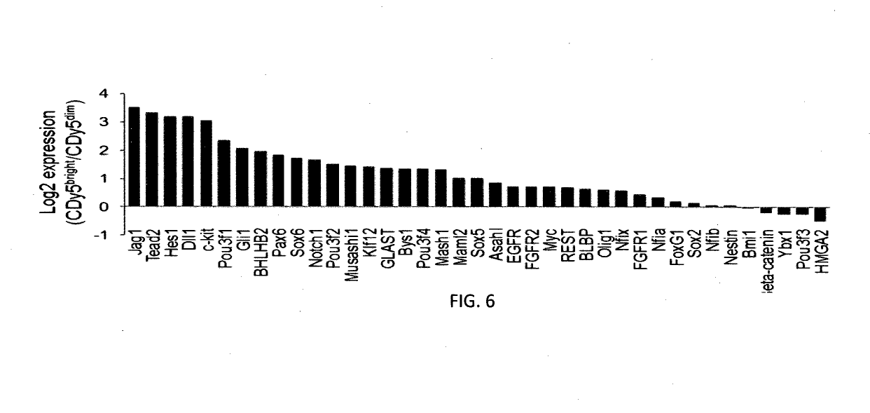

The expression levels of most genes analyzed, including Asah1, were higher in CDy5^{bright} cells compared to CDy5^{dim} cells. Noticeably, expressions of the genes directly involved in Notch signaling, such as Jag1, Dll1 and Hes1 (17), were particularly higher in CDy5^{bright} cells (FIG. 6).

### Acid Ceramidase is Critical in Neurosphere Formation

To assess the effects of CDy5 and AC inhibitors on neurosphere formation, dissociated neurosphere cells were plated in 12-well culture plates at a density of 1,000 cells per well and cultures in the presence of 2 μM or 4 μM CDy5 or 0.01 to 10 μM AC inhibitor. For vehicle control, DMSO was added to a volume of 0.1%. The IC₅₀ values were calculated using GraphPad Prism software.

To specifically investigate the role of AC in neurosphere formation, dissociated neurosphere cells were treated with AC inhibitors Carmofur and Ceranib-2 in concentrations ranging from 0.01 to 10 Carmofur is an established anti-neoplastic drug used for the treatment of gastrointestinal and breast cancers (18,19), but its anti-proliferative effect mediated by specific inhibition of AC has only been recently revealed (20). Ceranib-2 was developed as a ceramidase inhibitor by screening ˜50,000 small molecules and chemical optimization of a lead compound. It inhibits cancer cell growth in vivo as well as in vitro (21). When the numbers of neurospheres generated in the presence of these inhibitors were counted, significant inhibition of neurosphere formation was observed, with IC₅₀ s of 0.92 μM for Carmofur and 0.78 μM for Ceranib-2.

As the AC inhibitors reduced neurosphere formation, whether CDy5 exerts adverse effects on the proliferation of neurospheres was examined by culturing neurospheres in the presence of CDy5. The numbers of neurospheres grown in the medium containing 2 and 4 μM of CDy5 were 229±72 and 228±83, respectively, which were not significantly different from 189±40 grown in the vehicle-added control group, suggesting that CDy5 does not affect normal proliferation and growth of neural stem cells.

### Cytotoxicity Assay

Whether CDy5 exerts adverse effects on proliferation and differentiation of neural stem cells was also assessed using a neurosphere assay and multipotency test. For cytotoxicity assay, dissociated neurosphere cells were plated in triplicate in 6-well culture plates at a density of 1,000 cells per well and cultured in the presence of 2 μM or 4 μM CDy5. For vehicle control, 0.1% DMSO was added. After six days, the number of neurospheres was determined.

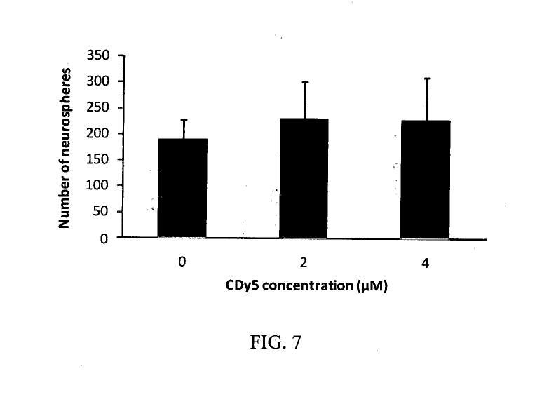

FIG. 7 is a bar graph and shows the number of neurospheres counted after six days of culture in medium containing the indicated concentration of CDy5. The number of neurospheres passaged and grown in the presence of 2 μM and 4 μM CDy5 was not significantly different from the vehicle-added control group.

The single neurospheres generated in the presence of CDy5 and normal medium were then plated onto glass coverslips coated with laminin and poly-L-lysine, cultured in medium containing 5% fetal bovine serum and allowed to undergo differentiation. The differentiated cells were immunostained using antibodies raised against markers of astrocyte, neuron and oligodendrocyte to classify the neurospheres into uni-, bi- and tri-potent depending on the number of positively stained cell types. The control group contained 55±6% unipotent neurospheres, 29±3% bipotent neurospheres and 16±3% tripotent neurospheres. The CDy5 group contained 49±1% unipotent neurospheres, 31±4% bipotent neurospheres and 20±3% tripotent neurospheres. These are very similar ratios of differentiation potential of neurospheres between control and CDy5 groups. Taken together, the results of proliferation and differentiation experiments imply that CDy5 does not affect normal function and growth of neural stem cells.

Cell type specific staining by an imaging probe may be due to a higher level of target molecule that specifically interacts with the probe in the cells and physicochemical properties of the probe that make the interaction strong and stable. CDy5 was among the several compounds identified in a primary screening that stained a distinct cell population in neurospheres. The chloroacetamide group of CDy5 can form a covalent bond with a nucleophilic amino acid functional group such as thiol of cysteine or primary amine of lysine in the binding target protein. This property is particularly useful for the identification of the binding target, since the fluorescence signal can be traced during in vitro analysis. For a long-term imaging of cells, especially for rapidly dividing cells, the stability of fluorescently labeled target protein is also a critical factor. It is been known that mature form of AC is not secreted out of the cell and its half life is longer than 20 hours (15). Although not wishing to be bound by any particular theory, the long time-lapse imaging of proliferating neurospheres using CDy5 even over 2 days might be possible due to the strong interaction between CDy5 and AC and the exceptional stability of AC.

AC hydrolyzes ceramide into fatty acid and sphingosine at a pH of about 4.5 (22,23), and is highly active, particularly in kidney and brain among the organs of a mouse (14). Deficiency of this enzyme activity causes systemic accumulation of ceramide leading to a lysosomal lipid storage disorder known as Faber disease (24,25). In embryonic development, the AC gene Asah1 starts to be expressed from the two-cell stage, and if the gene is completely knocked out, the two-cell embryo does not divide but undergoes apoptotic death (26). On the other hand, increased expression of AC in proliferative and more drug resistant cancer cells has been recently reported (27-29). Sphingosine-1-phosphate (S1P), which is readily generated from ceramide by ceramidases and sphingosine kinases, has been known as an intracellular second messenger involved in cell proliferation (30,31) as well as a G-protein coupled receptor ligand (32,33). Although TNF receptor-associated factor 2 and histone deacetylase have been identified as binding targets of intracellular SIP (34,35), the mechanism of cell proliferation by S1P is not understood. AC also has been known to be functionally important for cancer cell proliferation and has hence been proposed as an attractive target for cancer therapy (36,37). Our results from single cell gene expression analysis and neurosphere assay with AC inhibitors imply that AC is one of the important molecules highly expressed to render the neural stem cells more proliferative in the early stage of neurosphere formation. Whether or not AC is critical also for the proliferation of other types of stem cells remains to be investigated.

### REFERENCES

1 Morrison, S. J. & Kimble, J. Asymmetric and symmetric stem-cell divisions in development and cancer. Nature 441, 1068-1074, doi:10.1038/nature04956 (2006).
2 Neumuller, R. A. & Knoblich, J. A. Dividing cellular asymmetry: asymmetric cell division and its implications for stem cells and cancer. Genes & development 23, 2675-2699, doi:10.1101/gad.1850809 (2009).
3 Suslov, O. N., Kukekov, V. G., Ignatova, T. N. & Steindler, D. A. Neural stem cell heterogeneity demonstrated by molecular phenotyping of clonal neurospheres. Proceedings of the National Academy of Sciences of the United States of America 99, 14506-14511, doi:10.1073/pnas.212525299 (2002).
4 Narayanan, G. et al. Single-cell mRNA profiling identifies progenitor subclasses in neurospheres. Stem Cells Dev., doi:10.1089/scd.2012.0232 (2012).
5 Gonczy, P. Mechanisms of asymmetric cell division: flies and worms pave the way. Nat. Rev. Mol. Cell. Biol. 9, 355-366, doi:10.1038/nrm2388 (2008).
6 Mayer, B., Emery, G., Berdnik, D., Wirtz-Peitz, F. & Knoblich, J. A. Quantitative analysis of protein dynamics during asymmetric cell division. Curr Biol 15, 1847-1854, doi:10.1016/j.cub.2005.08.067 (2005).
7 Ohtsuka, T. et al. Visualization of embryonic neural stem cells using Hes promoters in transgenic mice. Molecular and cellular neurosciences 31, 109-122, doi:10.1016/j.mcn.2005.09.006 (2006).
8 Kang, N. Y., Ha, H. H., Yun, S. W., Yu, Y. H. & Chang, Y. T. Diversity-driven chemical probe development for biomolecules: beyond hypothesis-driven approach. Chem. Soc. Rev. 40, 3613-3626, doi:10.1039/c0cs00172d (2011).
9 Im, C. N. et al. A fluorescent rosamine compound selectively stains pluripotent stem cells. Angew. Chem. Int. Ed. Engl. 49, 7497-7500, doi:10.1002/anie.201002463 (2010).
10 Lee, S. C. et al. Development of a fluorescent chalcone library and its application in the discovery of a mouse embryonic stem cell probe. Chem. Commun. (Camb) 48, 6681-6683, doi:10.1039/c2cc31662e (2012).
11 Yun, S. W. et al. Neural stem cell specific fluorescent chemical probe binding to FABP7. Proc. Natl. Acad. Sci. U.S.A. 109, 10214-10217, doi:10.1073/pnas.1200817109 (2012).
12 Kang, N. Y., Ha, H. H., Yun, S. W., Yu, Y. H. & Chang, Y. T. Diversity-driven chemical probe development for biomolecules: beyond hypothesis-driven approach. Chemical Society reviews 40, 3613-3626, doi:10.1039/c0cs00172d (2011).
13 Koch, J. et al. Molecular cloning and characterization of a full-length complementary DNA encoding human acid ceramidase. Identification Of the first molecular lesion causing Farber disease. The Journal of biological chemistry 271, 33110-33115 (1996).
14 Li, C. M. et al. Cloning and characterization of the full-length cDNA and genomic sequences encoding murine acid ceramidase. Genomics 50, 267-274, doi:10.1006/geno. 1998.5334 (1998).
15 Ferlinz, K. et al. Human acid ceramidase: processing, glycosylation, and lysosomal targeting. The Journal of biological chemistry 276, 35352-35360, doi:10.1074/jbc.M103066200 (2001).
16 Ramasamy, S., Narayanan, G., Sankaran, S., Yu, Y. H. & Ahmed, S. Neural stem cell survival factors. Archives of biochemistry and biophysics 534, 71-87, doi:10.1016/j.abb.2013.02.004 (2013).
17 Lathia, J. D., Mattson, M. P. & Cheng, A. Notch: from neural development to neurological disorders. Journal of neurochemistry 107, 1471-1481, doi:10.1111/j.1471-4159.2008.05715.x (2008).
18 Sakamoto, J. et al. An individual patient data meta-analysis of adjuvant therapy with carmofur in patients with curatively resected colon cancer. Japanese journal of clinical oncology 35, 536-544, doi:10.1093/jjco/hyi147 (2005).
19 Morimoto, K. & Koh, M. Postoperative adjuvant use of carmofur for early breast cancer. Osaka city medical journal 49, 77-83 (2003).
20 Realini, N. et al. Discovery of highly potent acid ceramidase inhibitors with in vitro tumor chemosensitizing activity. Scientific reports 3, 1035, doi:10.1038/srep01035 (2013).
21 Draper, J. M. et al. Discovery and evaluation of inhibitors of human ceramidase. Molecular cancer therapeutics 10, 2052-2061, doi:10.1158/1535-7163.MCT-11-0365 (2011).
22 Gatt, S. Enzymatic hydrolysis of sphingolipids. I. Hydrolysis and synthesis of ceramides by an enzyme from rat brain. The Journal of biological chemistry 241, 3724-3730 (1966).
23 Okino, N. et al. The reverse activity of human acid ceramidase. The Journal of biological chemistry 278, 29948-29953, doi:10.1074/jbc.M303310200 (2003).
24 Sands, M. S. Farber disease: understanding a fatal childhood disorder and dissecting ceramide biology. EMBO molecular medicine 5, 799-801, doi:10.1002/emmm.201302781 (2013).
25 Sugita, M., Dulaney, J. T. & Moser, H. W. Ceramidase deficiency in Farber's disease (lipogranulomatosis). Science 178, 1100-1102 (1972).
26 Eliyahu, E., Park, J. H., Shtraizent, N., He, X. & Schuchman, E. H. Acid ceramidase is a novel factor required for early embryo survival. FASEB journal: official publication of the Federation of American Societies for Experimental Biology 21, 1403-1409, doi:10.1096/fj.06-7016com (2007).
27 Bedia, C., Casas, J., Andrieu-Abadie, N., Fabrias, G. & Levade, T. Acid ceramidase expression modulates the sensitivity of A375 melanoma cells to dacarbazine. The Journal of biological chemistry 286, 28200-28209, doi:10.1074/jbc.M110.216382 (2011).
28 Ramirez de Molina, A. et al. Acid ceramidase as a chemotherapeutic target to overcome resistance to the antitumoral effect of choline kinase alpha inhibition. Current cancer drug targets 12, 617-624 (2012).
29 Saad, A. F. et al. The functional effects of acid ceramidase overexpression in prostate cancer progression and resistance to chemotherapy. Cancer biology & therapy 6, 1455-1460 (2007).
30 Zhang, H. et al. Sphingosine-1-phosphate, a novel lipid, involved in cellular proliferation. The Journal of cell biology 114, 155-167 (1991).
31 Olivera, A. & Spiegel, S. Sphingosine-1-phosphate as second messenger in cell proliferation induced by PDGF and FCS mitogens. Nature 365, 557-560, doi:10.1038/365557a0 (1993).
32 Meyer zu Heringdorf, D. et al. Sphingosine kinase-mediated Ca2+ signalling by G-protein-coupled receptors. The EMBO journal 17, 2830-2837, doi:10.1093/emboj/17.10.2830 (1998).
33 Hobson, J. P. et al. Role of the sphingosine-1-phosphate receptor EDG-1 in PDGF-induced cell motility. Science 291, 1800-1803, doi:10.1126/science.1057559 (2001).
34 Alvarez, S. E. et al. Sphingosine-1-phosphate is a missing cofactor for the E3 ubiquitin ligase TRAF2. Nature 465, 1084-1088, doi:10.1038/nature09128 (2010).
35 Hait, N. C. et al. Regulation of histone acetylation in the nucleus by sphingosine-1-phosphate. Science 325, 1254-1257, doi:10.1126/science.1176709 (2009).
36 Zeidan, Y. H. et al. Molecular targeting of acid ceramidase: implications to cancer therapy. Current drug targets 9, 653-661 (2008).
37 Camacho, L. et al. Acid ceramidase as a therapeutic target in metastatic prostate cancer. Journal of lipid research 54, 1207-1220, doi:10.1194/jlr.M032375 (2013).
38 Ghosh, K. K., Ha, H. H., Kang, N. Y., Chandran, Y. & Chang, Y. T. Solid phase combinatorial synthesis of a xanthone library using click chemistry and its application to an embryonic stem cell probe. Chem Commun (Camb) 47, 7488-7490, doi:10.1039/c1cc11962a (2011).
39 Ahmed, S. et al. Transcription factors and neural stem cell self-renewal, growth and differentiation. Cell adhesion & migration 3, 412-424 (2009).
40 Pastrana, E., Silva-Vargas, V. & Doetsch, F. Eyes wide open: a critical review of sphere-formation as an assay for stem cells. Cell stem cell 8, 486-498, doi:10.1016/j.stem.2011.04.007 (2011).

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A compound represented by the following structural formula: 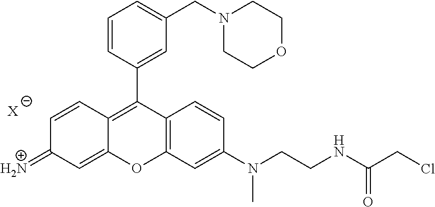
wherein X is an anion.

2. A method of detecting a neural stem cell in a sample, the method comprising:
contacting a sample potentially containing a neural stem cell with a compound having the structure of claim 1 under conditions sufficient to enable the compound to label the neural stem cell, if present; and
detecting a signal emitted by the compound,
thereby detecting the neural stem cell, if present, in the sample.

3. The method of claim 2, further comprising substantially removing unbound compound.

4. A method of distinguishing between a neural stem cell and a differentiated neural cell, the method comprising:
contacting a sample containing a neural stem cell and a differentiated neural cell with a compound having the structure of claim 1 under conditions sufficient to enable the compound to label the neural stem cell; and
detecting a signal emitted by the compound, wherein the presence of the signal indicates the neural stem cell,
thereby distinguishing between the neural stem cell and the differentiated neural cell.

5. The method of claim 4, further comprising substantially removing unbound compound from the sample.

6. A method of detecting acid ceramidase in a sample, the method comprising:
contacting a sample potentially containing acid ceramidase with a compound having the structure of claim 1 under conditions sufficient to enable the compound to label acid ceramidase, if present; and
detecting a signal emitted by the compound,
thereby detecting acid ceramidase, if present, in the sample.

7. The method of claim 6, further comprising substantially removing unbound compound from the sample.

8. A method of detecting a cell expressing acid ceramidase in a sample, the method comprising:
contacting a sample potentially containing a cell expressing acid ceramidase with a compound having the structure of claim 1 under conditions sufficient to enable the compound to label the cell expressing acid ceramidase, if present; and
detecting a signal emitted by the compound,
thereby detecting a cell expressing acid ceramidase, if present, in the sample.

9. The method of claim 8, further comprising substantially removing unbound compound from the sample.

10. The method of claim 8, wherein the cell is a neural stem cell.

11. A method of detecting symmetric and asymmetric division of a neural stem cell into a first daughter cell and a second daughter cell, the method comprising:
contacting a sample containing a neural stem cell with a compound of Structural Formula (I): 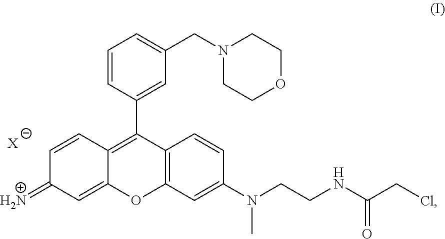
wherein X is an anion, under conditions sufficient to enable the compound of Structural Formula (I) to label the neural stem cell;
allowing the neural stem cell to undergo division into the first daughter cell and the second daughter cell; and
detecting a signal emitted by the compound of Structural Formula (I) in the first and second daughter cells, wherein signals of approximately equal intensities in the first and second daughter cells indicate symmetric division, and the presence of a substantially greater signal in the first daughter cell compared to the second daughter cell indicates asymmetric division,
thereby detecting symmetric and asymmetric division of the neural stem cell into the first daughter cell and the second daughter cell.

12. The method of claim 11, wherein the division is symmetric division.

13. The method of claim 11, wherein the division is asymmetric division.

14. The method of claim 11, further comprising stimulating neural stem cell differentiation by contacting the neural stem cell with an agent that stimulates neural stem cell differentiation.

15. The method of claim 11, the method comprising detecting the signal emitted by the compound of Structural Formula (I) in the first and second daughter cells using live-cell imaging.

16. The method of claim 11, further comprising substantially removing unbound compound of Structural Formula (I) from the sample.

17. A method of identifying a compound that inhibits or stimulates neural stem cell differentiation, the method comprising:
contacting a first sample containing a neural stem cell with a compound of Structural Formula (I): 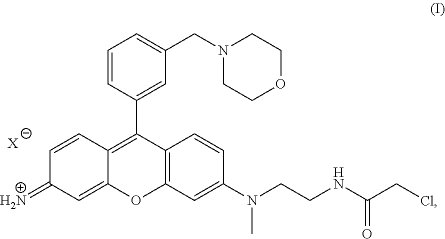
wherein X is an anion, and a compound that potentially inhibits or stimulates neural stem cell differentiation under conditions sufficient to enable the compound of Structural Formula (I) to label the neural stem cell;
incubating the neural stem cell under conditions sufficient to allow a neural stem cell in a second sample that has not been contacted with the compound that potentially inhibits or stimulates neural stem cell differentiation to undergo division into at least a first daughter cell and a second daughter cell; and
detecting a signal, if present, emitted by the compound of Structural Formula (I) in cells in the first and second samples, wherein a signal in a substantially different number of cells in the first sample than in the second sample indicates inhibition or stimulation of neural stem cell differentiation,
thereby identifying a compound that inhibits or stimulates neural stem cell differentiation.

18. The method of claim 17, further comprising substantially removing unbound compound of Structural Formula (I) from the first and second samples.

19. The method of claim 17, wherein the method is a method of identifying a compound that inhibits neural stem cell differentiation.

20. The method of claim 19, further comprising stimulating neural stem cell differentiation by contacting the neural stem cell in the sample with an agent that stimulates neural stem cell differentiation.

21. The method of claim 17, wherein the method is a method of identifying a compound that stimulates neural stem cell differentiation.

22. The method of claim 17, the method comprising detecting the signal emitted by the compound of Structural Formula (I) using flow cytometry or fluorescence image analysis.
